(19) United States
(12) Patent Application Publication
(10) Pub. No.: US 2021/0198346 A1
(43) Pub. Date: Jul. 1, 2021

(54) TREATMENT OF IMMUNE DISEASES BY MEANS OF THE ANTIBODY-MEDIATED NEUTRALIZATION OF SPECIFIC INTESTINAL BACTERIA

(71) Applicant: Fraunhofer-Gesellschaft zur Förderung der angewandten Forschung e.V., München (DE)

(72) Inventors: Christopher OELKRUG, Aschenberg (DE); Daniel Christoph WAGNER, Mainz (DE); Armin BRAUN, Isernhagen (DE); Christina HESSE, Osterode (DE)

(21) Appl. No.: 17/265,506

(22) PCT Filed: Aug. 2, 2019
(86) PCT No.: PCT/EP2019/070910

§ 371 Date: Feb. 2, 2021

(30) Foreign Application Priority Data
Aug. 3, 2018 (DE) 10 2018 213 030.2

(51) Int. Cl. C07K 16/12; A61K 45/06

(52) U.S. Cl. CPC C07K 16/1282; A61K 45/06; A61K 2039/505; C07K 2317/24; C07K 2317/33; C07K 2317/76

(57) ABSTRACT

An antibody or an antigen-binding fragment thereof binds to an antigen of the bacterium Candidatus savagella and inhibits the adhesion of the bacterium to intestinal epithelial cells; and/or depletes the bacterium. A drug has the antibody or an antigen-binding fragment thereof. A kit has the antibody or an antigen-binding fragment thereof for reduction of Th17 cell proliferation, Th17 cell differentiation or Th17 cell activity and/or inhibition of the formation of antibodies against endogenous antigens by B cells. The kit can contain an antibiotic. A method for producing the antibody involves immunizing chickens with an immunogenic peptide from an antigen of the bacterium Candidatus savagella, and recovering and purifying the antibodies formed in the chickens or in an egg laid by said chickens. A method for producing the drug involves producing the antibody or an antigen-binding fragment thereof, and formulating the antibody or an antigen-binding fragment thereof as a drug.

### PRIORITY AND CROSS REFERENCE TO RELATED APPLICATIONS

This application is the U.S. National Phase application under 35 U.S.C. § 371 of International Application No. PCT/EP2019/070910, filed Aug. 2, 2019, designating the U.S. and published as WO 2020/025801 A1 on Feb. 6, 2020, which claims the benefit of German Application No. DE 10 2018 213 030.2, filed Aug. 3, 2018. Any and all applications for which a foreign or a domestic priority is claimed is/are identified in the Application Data Sheet filed herewith and is/are hereby incorporated by reference in their entireties under 37 C.F.R. § 1.57.

### SEQUENCE LISTING IN ELECTRONIC FORMAT

The present application is being filed along with an Electronic Sequence Listing as an ASCII text file via EFS-Web. The Electronic Sequence Listing is provided as a file entitled HRZGO01012APCSEQLIST.txt, created and last saved on Feb. 2, 2021, which is 6,246 bytes in size. The information in the Electronic Sequence Listing is incorporated herein by reference in its entirety.

### FIELD

The present invention relates to the treatment of immune diseases and other diseases through the antibody-mediated neutralization of specific intestinal bacteria.

### SUMMARY

The present invention relates to the treatment of immune diseases and other diseases through the antibody-mediated neutralization of specific intestinal bacteria. More particularly, the invention relates to antibody or an antigen-binding fragment thereof, wherein the antibody or the antigen-binding fragment binds to an antigen of the bacterium Candidatus savagella and (i) inhibits the adhesion of the bacterium to intestinal epithelial cells, preferably human intestinal epithelial cells, and/or (ii) depletes the bacterium. The invention further provides a drug comprising the antibody according to the invention or an antigen-binding fragment thereof or comprising an antibody which has been produced by the method according to the invention. The invention furthermore relates to a kit comprising an antibody according to the invention or an antigen-binding fragment thereof for reduction of Th17 cell proliferation, Th17 cell differentiation or Th17 cell activity and/or inhibition of the formation of antibodies against endogenous antigens by B cells. The kit according to the invention optionally contains an antibiotic. The invention also provides a method for producing an antibody according to the invention, the method comprising: a) immunizing chickens with an immunogenic peptide from an antigen of the bacterium Candidatus savagella; and b) recovering and purifying the antibodies formed in the chickens or in an egg laid by said chickens. The invention lastly relates to a method for producing a drug according to the invention, comprising: a) producing an antibody according to the invention or an antigen-binding fragment thereof; and b) formulating the antibody or an antigen-binding fragment thereof as a drug.

### BRIEF DESCRIPTION OF THE DRAWINGS

FIG. 1 shows a graphic summary of an embodiment of the concept forming the basis of the present invention: Specific filamentous Candidatus savagella bacteria (segmented filamentous bacteria, SFB) colonize the intestinal wall and, via dendritic cells (DC), activate Th17 cells, which contribute to autoimmunity and atopy. First, suitable bacterial wall proteins of said bacterium are identified (1), synthesized and injected into chickens (2). The chickens form highly specific anti-SFB antibodies, which can be isolated from the eggs (3) and are available for oral antibody therapy in humans (4). A reduction in the SFB microbial count in the intestines can lead to a reduction in Th17 effector cell activity and thus to immunotolerance.

### DETAILED DESCRIPTION

Every person harbors more than 100 trillion bacteria, the entirety of which is referred to as the microbiome. Most of these bacteria colonize the intestines and are useful to humans in the digestion of plant fibers, the provision of vitamins and the displacement of harmful microorganisms. This symbiosis represents a daily test for the human immune system: only a monolayer barrier of epithelial cells separates humans from their microbiome. The immune cells of the intestinal wall constantly have to decide between tolerance to useful bacteria and defense against harmful bacteria. It is therefore not surprising that the composition of the microbiome has a direct and significant influence on the immune system and thus on human health [1].

In the past few decades, there has been a significant rise in immune-mediated diseases in all industrialized countries. These include the frequent diagnoses of ulcerative colitis, Crohn's disease, rheumatoid arthritis, type 1 diabetes mellitus and multiple sclerosis, but also atopic diseases, such as neurodermatitis and allergic asthma. The rate at which these diseases are increasing is so rapid that genetic changes cannot be the cause. For example, the incidence of allergic asthma in the USA rose by 75% between 1980 and 1994, whereas the incidence in developing countries remained unchanged over the same period [6]. The question that must be asked is what environmental influence can explain this rapid rise in immune-mediated diseases. Altered eating habits, especially the increased intake of salt, fat, sugar and pesticide- and antibiotic-contaminated foods, but also the absence of immunomodulating, parasitic intestinal worms, may cause a disturbed homeostasis between microbiome and immune system (dysbiosis). Other causes of dysbiosis may lie in altered intestinal colonization after birth, for example after caesarean section, and be due to increased hygiene in the living environment. However, the human microbiome has a considerable influence on the manifestation of immune reactions [1]. Certain bacteria which act nonpathologically per se can still have an immunoregulatory or immunostimulating influence. Influencing the microbiome with the aim of therapeutic immunomodulation therefore represents, incidence of selected immune diseases [7], an interesting therapeutic approach for numerous socioeconomically relevant diseases. Although previous approaches such as probiotic [8], prebiotic [9] and antibiotic [10] therapies have been successful in some cases, they are too unspecific for achieving the desired immunological effect.

A filamentous bacterium called Candidatus savagella (segmented filamentous bacteria, SFB) exhibits a particularly interesting interaction with its host: during infancy, it is substantially involved in the development and maturation of the immune system; in later stages of life, it stimulates certain effector cells of the acquired immune system, the Th17 cells, which in turn can fight harmful intestinal bacteria. However, the same immune cells are also responsible for the development and progression of immune-mediated diseases such as autoimmune diseases and atopies. In fact, test animals without SFB colonization exhibit distinctly fewer symptoms in animal models of rheumatoid arthritis, multiple sclerosis and allergic asthma [2-4].

The bacterium Candidatus savagella (SFB) generates a continuous physiological inflammatory reaction in the intestinal mucosa, which altogether leads to a strengthening of the immune defense against pathological intestinal germs. This evolutionarily conserved concept can be harmful in excess (e.g., in the case of dysbiosis) and lead to immune-mediated diseases in the rest of the body [1]. In recent years, this connection has been of increasing interest in basic science and has been the subject of high-ranking publications [2; 3; 11-13].

US 2012/276149 describes the administration of SFB to strengthen immunocompetence against intestinal infections.

WO 2011/047153 describes a modulation of the Th17 immune response, including by influencing the adherence and proliferation of SFB.

However, a specific therapeutic concept has not yet been described.

The technical problem addressed by the present invention can therefore be considered that of providing such a therapeutic approach.

The technical problem is solved by the embodiments described in the claims and described below.

The invention therefore relates to an antibody or an antigen-binding fragment thereof, wherein the antibody or the antigen-binding fragment binds to an antigen of the bacterium Candidatus savagella and (i) inhibits the adhesion of the bacterium to intestinal epithelial cells, preferably human intestinal epithelial cells, and/or (ii) depletes the bacterium. Preferably, the antibody or the antigen-binding fragment binds to an antigen of the bacterium Candidatus savagella and inhibits the adhesion of the bacterium to intestinal epithelial cells, preferably human intestinal epithelial cells, and depletes the bacterium.

An antibody according to the invention or an antigen-binding fragment thereof binds specifically to an antigen of the bacterium Candidatus savagella (segmented filamentous bacteria, SFB), preferably to a bacterial wall protein.

Here, the antigen is chosen such that the binding of the antibody according to the invention or the antigen-binding fragment thereof inhibits the adhesion of the bacterium to intestinal epithelial cells or depletes the bacterium. The binding of the antibody according to the invention or the antigen-binding fragment thereof to the antigen can also inhibit the proliferation of the bacterium. Combinations of these mechanisms are also encompassed in the context of the invention. For example, the antigen can be selected such that the antibody according to the invention binds the antigen of the bacterium Candidatus savagella and inhibits the adhesion of the bacterium to intestinal epithelial cells and depletes the bacterium. Alternatively, the antigen can be chosen such that the antibody according to the invention binds the antigen of the bacterium Candidatus savagella and inhibits the adhesion of the bacterium to intestinal epithelial cells and inhibits the proliferation of the bacterium. A combination of all three mechanisms is also encompassed, and so the antigen is defined such that the antibody according to the invention or the antigen-binding fragment binds to the antigen of the bacterium Candidatus savagella and inhibits the adhesion of the bacterium to intestinal epithelial cells, inhibits the proliferation of the bacterium and depletes the bacterium.

In a study carried out at Fraunhofer IZI and ITEM, it was possible to show that the colonization density of SFB can be reduced by an oral antibody treatment and that this specific correction of the microbiome leads to an immunotolerance in the context of immune-mediated diseases. The SFB-specific antibodies were recovered from the eggs of chickens immunized with SFB proteins; this method allows the rapid and highly cost-effective generation of strongly binding antibodies that also exhibit an exceptional acid stability and are therefore highly suitable for an oral treatment [5].

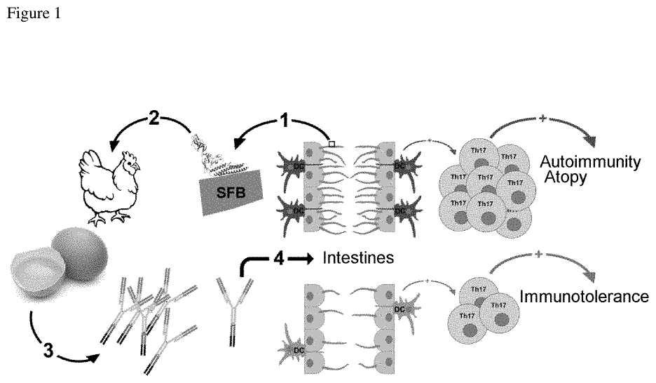

The concept forming the basis of the present invention is illustrated in FIG. 1: filamentous Candidatus savagella bacteria (segmented filamentous bacteria, SFB) colonize the intestinal wall and, via dendritic cells, activate Th17 cells, which contribute to autoimmunity and atopy. First, suitable bacterial wall proteins of said bacterium are identified, synthesized and injected into chickens. The chickens form highly specific, neutralizing anti-SFB antibodies, which can be isolated from the eggs and are available for oral antibody therapy. A reduction in the SFB microbial count in the intestines can lead to a reduction in Th17 effector cell activity and thus to immunotolerance.

A specific neutralization of SFB with orally administered antibodies has the major advantage that there is practically no transfer of the antibodies into the circulation and thus common adverse effects of an antibody therapy can be avoided, or adverse effects as observed for instance in the case of dexamethasone for allergic respiratory disease. Examples of the successful oral use of antibodies for the reduction of intestinal pathogenic viruses [14], fungi [15] and bacteria [16; 17] have already been described. However, oral antibody therapies with the aim of immunomodulation have so far only been carried out with the direct target of the immune system. A successful example thereof is the use of antibodies against the T cell surface protein CD3 ([18]; U.S. Pat. No. 7,883,703 B2).

A major advantage of this invention is the use of therapeutic IgY antibodies from the eggs of immunized chickens. This method has numerous advantages: besides a highly cost-effective production process, the antibodies can be produced rapidly and in large amounts. IgYs are more acid-resistant than IgGs and are therefore particularly highly suitable for oral administration. One chicken can produce up to 30 g of pure antibody per year, which antibody moreover exhibits a high binding strength. Lastly, IgYs have an Fc region different from mammalian IgGs; therefore, there are no adverse effects due to an activation of the recipient complement system [5].

In a “proof-of-concept” experiment, it has already been possible to demonstrate that the oral administration of anti-SFB IgY antibodies leads to a distinct reduction in SFB excretion—as a correlate of intestinal SFB colonization. Indications for the therapeutic relevance of this microbiome change were ultimately observed in the animal model of allergic respiratory disease (Fraunhofer ITEM): the reduction in intestinal SFB colonization led to a weakening of inflammatory cell infiltration into the lungs, to a comparable extent to the standard therapeutic dexamethasone.

An IgY antibody-mediated inhibition of the bacterium Candidatus savagella (SFB) can have a direct influence on immune-mediated diseases. Here, immune diseases such as multiple sclerosis, rheumatoid arthritis and allergic asthma are substantially determined by the activity of Th17 immune cells. But also other immune diseases or oncological diseases determined by Th17 activity can be treated preventively or therapeutically by a depletion of SFB.

The use of these IgY antibodies is conceivable as medicament for the prevention or therapy of immune-mediated diseases, especially Th17-dependent diseases. What is intended here is a therapeutic for microbiome correction. It can also be used as functional food or nutraceuticals, and so there may be possibilities for a greatly simplified authorization procedure, for example as novel food.

The antibodies according to the invention are preferably used for an oral administration in humans.

The bacterium “Candidatus savagella” (segmented filamentous bacteria, SFB) has, for example, been described by Schnupf et al. (Curr Opin Microbiol. 2017 February; 35: 100-109. doi: 10.1016/j.mib.2017.03.004. Epub 2017 Apr. 25; Semin Immunol. 2013 Nov. 30; 25(5): 342-51. doi: 10.1016/j.smim.2013.09.001. Epub 2013 Oct. 31) and also in US 2012/276149 and WO 2011/047153.

The “antigen” of the bacterium Candidatus savagella that is specifically bound by the antibody according to the invention or the antigen-binding fragment of the antibody is involved in the adhesion of the bacterium to intestinal epithelial cells, preferably human intestinal epithelial cells, the proliferation of the bacterium, and/or mediates a depletion, i.e., a killing, of the bacterium. A suitable antigen from Candidatus savagella bacteria is selected from the group of function-determining and segmented filamentous bacteria (SFB)-specific proteins. Such a protein is, for instance, characterized in that it is located at the bacterial wall (bacterial wall protein) and has an essential or even unique role in adherence to the intestinal epithelium and/or the survival of Candidatus savagella or segmented filamentous bacteria (SFB).

A suitable antigen from said bacterium is, for example, the myosin-cross-reactive antigen (MCRA) protein described in detail below, the amino acid sequence of which is shown in SEQ ID No. 1. Specific epitopes from the myosin-cross-reactive antigen (MCRA) protein encompass, for example, the amino acid sequence SVLDEFYWLDKKDPYSL (SEQ ID No. 2), PDFKAVRFTRRNQYESMI (SEQ ID No. 3) and QATSIKILRDGKEEEIKL (SEQ ID No. 4).

“Specific binding” of the antibody according to the invention or a fragment thereof to an antigen from the bacterium Candidatus savagella describes the binding properties of the antibody, such as, for instance, binding affinity, binding specificity and binding avidity; see, for example, David J. King, Applications and Engineering of Monoclonal Antibodies, pp. 240 (1998). Detailed analyses of antigen-antibody interactions are, for example, possible with surface plasmon resonance (SPR). The kinetic characterization of the binding properties of antibodies and the antigens thereof is an essential requirement in order to be able to assess their applicability for various methods. Besides binding strength (affinity, KD value), the rate constants for association (kass) and dissociation (kdiss) are also determined. This also makes it possible to establish the rate of complex formation and complex disintegration. This information helps to assess the efficiency of the antibodies according to the invention in diagnostic, biotechnological or therapeutic applications and to optimize processes in their application. The terms and abbreviations mentioned in relation to specific binding are used with their standard meanings.

The term “antibody” encompasses both a polyclonal antibody and a monoclonal antibody (mAb), which can be modified as described below. The antibody binds specifically to an antigen of the bacterium Candidatus savagella and is preferably a neutralizing antibody.

“Neutralizing antibody” refers here to the inhibition of the adhesion or binding of the bacterium Candidatus savagella to (preferably human) intestinal epithelial cells, the inhibition of the proliferation of said bacterium and/or the depletion or killing thereof by the antibody.

“Neutralization” of the bacterium Candidatus savagella refers to at least 50%, 60%, 70% or 75%, preferably 80% or 85%, particularly preferably 90% or 95% inhibition of the adhesion or binding of said bacterium to intestinal epithelial cells, or of the inhibition of the proliferation of the bacterium, or that at least 50%, 60%, 70% or 75%, preferably 80% or 85%, particularly preferably 90% or 95% of the total number or population of the bacterium are depleted, as measured in in vitro tests.

“Modified antibody” refers to a protein encoded by a modified immunoglobulin-coding region, which protein can be obtained by expression in a selected host cell. Such modified antibodies include genetically engineered antibodies (e.g., chimeric, reshaped, humanized or vectorized antibodies) or antibody fragments, which lack all or part of a constant region of immunoglobulins, for example Fv, Fab or F(ab)₂ and the like.

“Modified immunoglobulin-coding region” refers to a nucleic acid sequence which encodes a modified antibody. If the modified antibody is a CDR-grafted or humanized antibody, the sequences which encode the complementarity-determining regions (CDRs) from a nonhuman immunoglobulin, for instance a chicken antibody, are inserted into a first immunoglobulin partner which comprises human variable framework sequences. Optionally, the first immunoglobulin partner is operatively linked to a second immunoglobulin partner, for instance in order to produce a bispecific antibody.

“First immunoglobulin partner” refers to a nucleic acid sequence which encodes a human framework region or variable region of a human immunoglobulin, in which the native (or naturally occurring) CDR-coding regions have been replaced by the CDR-coding regions of a donor antibody, for example a chicken antibody. The human variable region can be a heavy chain, a light chain (or both chains) of an immunoglobulin, an analog or functional fragments thereof. Such CDR regions that are situated within the variable region of antibodies (immunoglobulins) can be determined by methods known in the art. For example, Kabat et al. (Sequences of Proteins of Immunological Interest, 4th ed., U.S. Department of Health and Human Services, National Institutes of Health (1987)) disclose rules for locating CDRs. In addition, computer programs which are useful for identifying CDR regions/structures are known.

“Second immunoglobulin partner” refers to a different nucleotide sequence which encodes a protein or peptide to which the first immunoglobulin partner has been fused, i.e., has been operatively linked, in frame or by means of an optional conventional linker sequence. It is preferably an immunoglobulin gene. The second immunoglobulin partner can include a nucleic acid sequence which encodes the entire constant region for the same (i.e., homologous—the first and the second modified antibody originate from the same source) or additional (i.e., heterologous) antibody of interest. It can be a heavy chain or light chain of an immunoglobulin (or both chains as part of an individual polypeptide). The second immunoglobulin partner is not restricted to a particular immunoglobulin class or an isotype. In addition, the second immunoglobulin partner can comprise part of a constant region of an immunoglobulin, as is found in an Fab or F(ab)₂, i.e., a discrete part of a human constant region or framework region. Such a second immunoglobulin partner can also comprise a sequence which encodes an integral membrane protein which is exposed on the outer surface of a host cell, for example as part of a phage display library, or a sequence which encodes a protein for analytical or diagnostic detection, for example horseradish peroxidase, β-galactosidase, etc.

The terms Fv, Fc, Fd, Fab or F(ab)₂ are used with their standard meanings (see, for example, Harlow et al., Antibodies A Laboratory Manual, Cold Spring Harbor Laboratory (1988)).

As used here, a “genetically engineered antibody” describes a type of modified antibody, i.e., a full-length synthetic antibody (e.g., a chimeric, unshaped or humanized antibody as opposed to an antibody fragment), in which part of the variable domains of the light and/or heavy chain of a selected acceptor antibody has been replaced by analogous parts from one or more donor antibodies (e.g., chicken antibodies) which have specificity for the selected epitope. For example, such molecules can include antibodies which are characterized by a humanized heavy chain which is associated with an unmodified light chain (or chimeric light chain), or vice versa. Genetically engineered antibodies can also be characterized by modification of the nucleic acid sequences which encode the framework regions of the light and/or heavy variable domain of the acceptor antibody in order to maintain the binding specificity of a donor antibody, for example a chicken antibody. Said antibodies can encompass an exchange of one (e.g., CDR-H3) or more CDRs (preferably all CDRs, i.e., CDR-L1, CDR-L2, CDR-L3, CDR-H1, CDR-H2 and CDR-H3) from the acceptor antibody for CDRs from a donor antibody described here, i.e., a chicken antibody.

A “chimeric antibody” refers to a type of genetically engineered antibody which contains a naturally occurring variable region (light chain and heavy chains) that originates from a donor antibody (e.g., a chicken antibody) in association with constant regions of the light and heavy chain that originate from an acceptor antibody.

A “humanized antibody” refers to a type of genetically engineered antibody, the CDRs of which originate from a nonhuman donor immunoglobulin, for instance a chicken antibody, with the remaining immunoglobulin-originating parts of the molecule originating from one (or more) human immunoglobulins. In addition, framework residues can be modified in order to maintain binding affinity (see, for example, Queen et al., Proc. Natl. Acad. Sci. USA, 86: 10029-10032 (1989), Hodgson et al., Bio/Technology, 9:421 (1991)).

An agent can be bound to the antibody, for example in order to improve transport into the intestines. For example, the agent bound on the antibody can actively or passively influence transporter proteins in the intestines. The attachment can be chemical or, alternatively, the entity can be incorporated into the antibody by gene technology.

The term “donor antibody” refers to an antibody (monoclonal or recombinant) which contributes the nucleic acid sequences of its variable regions, CDRs or other functional fragments or analogs thereof for a first immunoglobulin partner in order to provide the modified immunoglobulin-coding region and the resulting expressed modified antibody with the antigenic specificity and neutralizing activity property of the donor antibody, for example the chicken antibody.

The term “acceptor antibody” refers to an antibody (monoclonal or recombinant) which is heterologous for the donor antibody and contributes all (or a portion, but preferably all) nucleic acid sequences which encode its framework regions of the heavy and/or light chain and/or its constant regions of the heavy and/or light chain for the first immunoglobulin partner. The acceptor antibody is preferably a human antibody.

“CDRs” are defined as the amino acid sequences of the complementarity-determining region of an antibody, which are the hypervariable regions of the heavy and light chains of an immunoglobulin. See, for example, Kabat et al., Sequences of Proteins of Immunological Interest, 4th ed., U.S. Department of Health and Human Services, National Institutes of Health (1987). There are three heavy-chain CDRs (CDR-H1, CDR-H2 and CDR-H3) and three light-chain CDRs (CDR-L1, CDR-L2 and CDR-L3) (or CDR regions) in the variable part of an immunoglobulin. As used here, “CDRs” refers to all three heavy-chain CDRs or all three light-chain CDRs (or both all heavy-chain CDRs and all light-chain CDRs, where appropriate). The structure and protein folding of the antibody may mean that other residues are regarded as part of the antigen binding region, and they would be understood in this way by a person skilled in the art. See, for example, Chothia et al. (1989), Conformations of immunoglobulin hypervariable regions; Nature 342, pp. 877-883.

CDRs provide the majority of the contact residues for the binding of the antibody to the antigen or epitope. CDRs of interest in this invention originate from the sequences of the variable heavy and light chain of the donor antibody, for example a chicken antibody, and include analogs of naturally occurring CDRs, the analogs sharing or retaining the same antigen-binding specificity and/or neutralizing ability as the donor antibody from which they were derived.

A “functional fragment” is a partial variable sequence of the heavy or light chain (e.g., minor deletions at the amino or carboxy terminus of the variable region of the immunoglobulin) that retains the same antigen-binding specificity and/or neutralizing ability as the antibody from which the fragment was derived.

An “analog” is an amino acid sequence modified with at least one amino acid, in which the modification can be chemical or a substitution or rearrangement of a few amino acids (i.e., preferably not more than 10, 9, 8, 7, 6, 5, 4, 3, 2 or 1 amino acid residue(s)), the modification of the amino acid sequence making it possible to maintain the biological properties, for example antigen specificity and high affinity, of the unmodified sequence. For example, (silent) mutations can be constructed via substitutions when particular endonuclease restriction sites are established within or around CDR-coding regions. The present invention contemplates the use of analogs of the antibody of the invention. It is commonly known that minor modifications of amino acid or nucleic acid sequences can, for example, lead to an allelic form of the original protein that retains substantially similar properties. Thus, analogs of the antibody of the invention include those in which the CDRs in the hypervariable region of the heavy and light chains are at least 80% homologous, preferably at least 85%, at least 90% homologous and particularly preferably at least 95%, 96%, 97%, 98% or 99% homologous to the CDRs as defined above as CDR-H1, CDR-H2, CDR-H3, CDR-L1, CDR-L2 and CDR-L3 and retain the specific binding to an antigen of the bacterium Candidatus savagella and neutralizing activity. Amino acid sequences are at least 80% homologous if they have 80% identical amino acid residues in a similar position when the sequences are optimally aligned, with gaps or insertions being counted as nonidentical residues. Algorithms for determination of sequence identity and programs for sequence comparison are well-known in the prior art.

Analogs can also appear as allelic variations. An “allelic variation or modification” is a change in the nucleic acid sequence. Such variations or modifications may be caused by the degeneracy of the genetic code or may be intentionally genetically engineered or recombinantly produced in order to provide the desired properties. Said variations and modifications may or may not lead to changes in an encoded amino acid sequence.

The term “effector agent” refers to nonprotein carrier molecules to which the modified antibodies and/or natural or synthetic light or heavy chains of the donor antibody, for instance a chicken antibody, or other fragments of the donor antibody can be associated by conventional means. Such nonprotein carriers can include conventional carriers which are used in the diagnostic field, for example polystyrene or other plastic beads, polysaccharides, for example as used in the BIAcore system [Pharmacia], or other nonprotein substances which are useful in the medical field and are safe for administration to humans and animals. Other effector agents can include a macrocycle for complexing a heavy metal atom or radioisotopes. Such effector agents can also be useful for increasing the half-life of the modified antibodies, for example polyethylene glycol (PEG).

A neutralizing antibody specific for the bacterium Candidatus savagella has not yet been described to date in the prior art and is made available for the first time by the present invention.

In a further aspect, the invention provides a pharmaceutical composition which comprises an antibody according to the invention together with a pharmaceutically acceptable diluent or carrier.

As is familiar to a person skilled in the art, it is possible to construct antibodies, modified antibodies and fragments by immunizing a nonhuman species (e.g., cattle, sheep, simian, chicken, rodent (e.g., mouse, hamster and rat), etc.) in order to generate a desirable immunoglobulin upon presentation with native or recombinant antigen from the bacterium Candidatus savagella from any species against which antibodies which are cross-reactive with respect to the native or recombinant antigen from the bacterium Candidatus savagella can be generated, for example human or chicken. Conventional hybridoma techniques are used in order to provide a hybridoma cell line which secretes a nonhuman monoclonal antibody, for instance a chicken antibody, against the native antigen from the bacterium Candidatus savagella. Such hybridomas are then screened for binding using native or recombinant antigen from the bacterium Candidatus savagella that has been applied to 384- or 96-well plates, wherein biotinylated native or recombinant antigen from the bacterium Candidatus savagella has been bound to a streptavidin-coated plate, or in a homogeneous europium-APC-coupled immunoassay using biotinylated native or recombinant antigen from the bacterium Candidatus savagella.

A native human antibody can, for example, be generated in a human antibody mouse such as the “Xenomouse” (Abgenix), in which the mouse immunoglobulin genes have been removed and genes which encode the human immunoglobulins have been inserted into the mouse chromosome. The mice are immunized normally and develop an antibody reaction which originates from the human genes. Thus, the mouse generates human antibodies while bypassing the need for humanization after selection of positive hybridomas (see L. L. Green, J. Immunol. Methods, 10 Dec. 1999; 231 (1-2):11-23).

An Fab fragment contains the entire light chain and the amino-terminal part of the heavy chain; and an F(ab′)2 fragment is the fragment formed by two Fab fragments bound by disulfide bonds. Fab fragments and F(ab′)2 fragments can be obtained by conventional means, for example cleavage of monoclonal antibodies (mAb) with the appropriate proteolytic enzymes, papain and/or pepsin, or by recombinant methods. The Fab and F(ab′)2 fragments are themselves useful as therapeutic or prophylactic and as donors for sequences which include the variable regions and CDR sequences which are useful in the formation of recombinant or humanized antibodies as described here.

The Fab and F(ab′)₂ fragments can also be constructed via a combinatorial phage library (see, for example, Winter et al., Ann. Rev. Immunol., 12: 433-455 (1994)) or via immunoglobulin chain exchange (chain shuffling) (see, for example, Marks et al., Bio Technology, 10: 779-783 (1992)).

Thus, human antibody fragments (Fv, scFv, Fab) which are specific for a native or recombinant antigen from the bacterium Candidatus savagella can be isolated using human antibody fragment phage display libraries. A library of bacteriophage particles which represent the human antibody fragment proteins is tested against the native or recombinant antigen from the bacterium Candidatus savagella. Those phages which represent antibody fragments which bind the native or recombinant antigen from the bacterium Candidatus savagella are retained from the library and amplified by cloning. The human antibody genes are then cut out of the specific bacteriophages and inserted into human IgG expression constructs which contain the constant regions of human IgG, in order to form the intact human IgG molecule having the variable regions from the isolated bacteriophage specific for native or recombinant antigen from the bacterium Candidatus savagella.

The donor antibodies can contribute sequences, such as, for example, peptide sequences of the variable heavy and/or light chain, framework sequences, CDR sequences, functional fragments and analogs thereof, and the nucleic acid sequences encoding them, which sequences are useful in constructing and obtaining various modified antibodies which are characterized by the antigen-binding specificity of the donor antibody, for example a chicken antibody.

Taking account of the degeneracy of the genetic code, it is possible to construct various coding sequences encoding the amino acid sequences of the variable heavy and light chain and CDR sequences and also functional fragments and analogs thereof that share the antigen specificity of the donor antibody, for instance a chicken antibody. Isolated nucleic acid sequences or fragments thereof that encode the peptide sequences of the variable chain or CDRs can be used to generate modified antibodies, for example chimeric or humanized antibodies, or other genetically engineered antibodies if they are operatively combined with a second immunoglobulin partner.

Modified immunoglobulin molecules can encode modified antibodies, which include genetically modified antibodies such as chimeric antibodies and humanized antibodies. A desired modified immunoglobulin-coding region contains CDR-coding regions which encode peptides having the antigen specificity of an anti-Candidatus savagella antigen antibody, preferably a high-affinity antibody, inserted into a first immunoglobulin partner (a human framework region or variable region of a human immunoglobulin).

Preferably, the first immunoglobulin partner is operatively linked to a second immunoglobulin partner. The second immunoglobulin partner is defined above and can include a sequence which encodes a second antibody region of interest, for example an Fc region. Second immunoglobulin partners can also include sequences which encode another immunoglobulin to which the constant region of the light or heavy chain has been fused in frame or by means of a linker sequence. Genetically engineered antibodies which are directed against functional fragments or analogs of native or recombinant antigen from the bacterium Candidatus savagella can be constructed to bring about increased binding.

The second immunoglobulin partner can also be associated with effector agents as defined above, including nonprotein carrier molecules to which the second immunoglobulin partner can be operatively bound by conventional means.

The fusion or binding between the second immunoglobulin partners, for example antibody sequences, and the effector agent can be achieved by any suitable means, for example by conventional covalent or ionic bonds, protein fusions or heterobifunctional crosslinkers, for example carbodiimide, glutaraldehyde and the like. Such techniques are known in the art and are described in general in conventional chemistry and biochemistry texts.

In addition, conventional linker sequences which simply provide a desired amount of space between the second immunoglobulin partner and the effector agent can also be constructed into the modified immunoglobulin-coding region. The construction of such linkers is commonly known to those skilled in the art.

In yet a further embodiment, the antibody can have an additional agent attached thereto. For example, the method of recombinant DNA technology can be used in order to generate a genetically engineered antibody in which the Fc fragment or the CH2-CH3 domain of a complete antibody molecule has been replaced by an enzyme or some other detectable molecule (i.e., a polypeptide effector or a reporter molecule).

The second immunoglobulin partner can also be operatively linked to a nonimmunoglobulin peptide, protein or fragment thereof which is heterologous in relation to the CDR-containing sequence having the antigen specificity of anti-Candidatus savagella antigen antibodies. The resulting protein can exhibit both anti-Candidatus savagella antigen specificity and properties of the nonimmunoglobulin when expressed. The property of the fusion partner can, for example, be a functional property such as another binding or receptor domain or a therapeutic property, if the fusion partner itself is a therapeutic protein, or additional antigenic properties.

Another desirable protein of this invention can be a complete full-length antibody molecule having heavy and light chains or any discrete fragment thereof such as the Fab or F(ab)₂ fragments, a heavy-chain dimer or any minimal recombinant fragments thereof such as an Fv or a single-chain antibody (SCA) or any other molecule having the same specificity as the selected donor antibody, for instance a chicken antibody. Such a protein can be used in the form of a modified antibody or can be used in its unfused form.

Whenever the second immunoglobulin partner originates from an antibody which differs from the donor antibody, for instance a chicken antibody, the result is a genetically engineered or recombinantly produced antibody. Genetically engineered or recombinantly produced antibodies can comprise immunoglobulin (Ig) constant regions and variable framework regions from a source, for example the acceptor antibody, and one or more (preferably all) CDRs from the donor antibody, for instance a chicken antibody. In addition, modifications, for example deletions, substitutions or additions, to the framework region of the light and/or heavy variable domain of the acceptor monoclonal antibody (mAb) at the nucleic acid or amino acid level or to the donor CDR regions can be carried out in order to maintain the antigen-binding specificity of the donor antibody, for instance a chicken antibody.

Such genetically engineered or recombinantly produced antibodies are constructed in order to use one (or both) of the variable heavy and/or light chains of the anti-Candidatus savagella antigen antibody or one or more of the CDRs of the heavy or light chain. The genetically engineered or recombinantly produced antibodies can be neutralizing as defined above.

Such genetically engineered or recombinantly produced antibodies can include a humanized antibody containing the framework regions of a selected human immunoglobulin or subtype or a chimeric antibody containing the constant regions of the human heavy and light chain, fused to the functional fragments of the anti-Candidatus savagella antigen antibody. A suitable human (or other animal) acceptor antibody can be one selected from a conventional database, for example the KABAT® database, the Los Alamos database and Swiss Protein database, by homology with the nucleotide and amino acid sequences of the donor antibody, for example a chicken antibody. A human antibody which is characterized by a homology with the framework regions of the donor antibody (on an amino acid basis) can be suitable for providing a heavy-chain constant region and/or a heavy-chain variable framework region for insertion of the donor CDRs. A suitable acceptor antibody which can donate light-chain constant or variable framework regions can be selected in a similar manner. It should be noted that the heavy and light chains of the acceptor antibody do not have to originate from the same acceptor antibody.

Desirably, the heterologous framework regions and the constant regions are selected from human immunoglobulin classes and isotypes such as IgG (subtypes 1 to 4), IgM, IgA and IgE. However, the acceptor antibody need not comprise only human immunoglobulin protein sequences. For example, it is possible to construct a gene in which a DNA sequence encoding part of a human immunoglobulin chain is fused to a DNA sequence encoding a nonimmunoglobulin amino acid sequence such as a polypeptide effector or a reporter molecule.

In a humanized antibody, the variable domains in both the human heavy and light chains were preferably genetically engineered by means of one or more CDR exchanges. It is possible to use all six CDRs or various combinations of fewer than the six CDRs. Preferably, all six CDRs are exchanged. It is possible to exchange the CDRs only in the human heavy chain, the light chain used being the unmodified light chain from the human acceptor antibody. Alternatively, a compatible light chain from another human antibody can be selected by using the conventional antibody databases. The rest of the genetically engineered antibody can originate from any suitable human acceptor immunoglobulin.

The genetically engineered or recombinantly produced humanized antibody thus has the structure of a natural human antibody or a fragment thereof and has the combination of properties which are necessary for an effective therapeutic use.

Those skilled in the art will understand that a genetically engineered antibody can be further modified by changes to the amino acids of the variable domain without necessarily affecting the specificity and high affinity of the donor antibody, for instance a chicken antibody (i.e., an analog). It is envisaged that amino acids of the heavy and light chain can be substituted by other amino acids in either the frameworks of the variable domain or CDRs, or both.

In addition, the constant region can be modified in order to increase or decrease selective properties of the molecules of the present invention, such as, for example, dimerization, binding to Fc receptors or the ability to bind and activate complement (see, for example, Angal et al., Mol. Immunol. 30: 105-108 (1993), Xu et al., J. Biol. Chem. 239: 3469-3474 (1994), Winter et al., EP 307,434-B).

A modified antibody which is a chimeric antibody differs from the humanized antibodies described above by providing the entire variable regions of the heavy chain and light chain of the nonhuman donor antibody, for instance a chicken antibody, including the framework regions, in association with the constant regions of the human immunoglobulin for both chains.

The production of antibodies which bind specifically to an antigen is well-described in the prior art, for example by the techniques described in Sambrook et al. (Molecular Cloning (A Laboratory Manual), 2nd edition, Cold Spring Harbor Laboratory (1989; 2001)). The production of a humanized monoclonal antibody is briefly outlined below. The heavy and light variable regions which contain at least the CDR-coding regions and those parts of the framework regions of the light and/or heavy variable domain of the acceptor mAb that are necessary for maintaining the binding specificity of the donor mAb, for instance a chicken monoclonal antibody, and also the remaining immunoglobulin-originating parts of the antibody chain which originate from a human immunoglobulin are obtained by using polynucleotide primers and reverse transcriptase. The CDR-coding regions are identified by using a known database and by comparison with other antibodies.

A chicken/human chimeric antibody can then be produced and tested for binding ability. Such a chimeric antibody contains the entire VH and VL regions of the nonhuman chicken donor antibody in association with the constant regions of human Ig for both chains.

Homologous framework regions of a variable region of the heavy chain from a human antibody can be identified by using computerized databases, for example KABAT®, and a human antibody with homology to the chicken donor antibody will be selected as the acceptor antibody. A suitable variable framework region of the light chain can be established in a similar manner.

A humanized antibody can originate from the chimeric antibody or be preferably produced synthetically by insertion of the CDR-coding regions of the chicken donor mAb from the heavy and light chains in an appropriate manner within the selected framework of the heavy and light chain. Alternatively, a humanized antibody can be produced by using standard mutagenesis techniques. Thus, the resulting humanized antibody contains human framework regions and CDR-coding regions of the chicken donor mAb. Framework residues can be subsequently manipulated. The resulting humanized antibody can be expressed in recombinant host cells, for example COS, CHO or myeloma cells.

A conventional expression vector or a recombinant plasmid is produced by placement of these coding sequences for the antibody in operative association with conventional regulatory control sequences which can control multiplication and expression in a host cell and/or secretion from a host cell. Regulatory sequences include promoter sequences, for example a CMV promoter, and signal sequences which can originate from other known antibodies. Similarly, a second expression vector can be produced using a DNA sequence which encodes a complementary light or heavy chain of an antibody. Preferably, said second expression vector is identical to the first one, except where the coding sequences and selectable markers are concerned, in order to ensure as far as possible that each polypeptide chain is functionally expressed. Alternatively, the coding sequences of the heavy and light chain for the modified antibody can be based on a single vector.

A selected host cell is cotransfected with both the first and the second vector (or simply transfected with a single vector) by conventional techniques in order to generate the transfected host cell comprising both the recombinant or synthetic light and heavy chains. The transfected cell is then cultured by conventional techniques in order to generate the genetically engineered or recombinantly produced antibody of the invention. The humanized antibody which includes the association of the recombinant heavy chain and/or light chain is screened from the culture by a suitable assay, for example ELISA or RIA. Similar conventional techniques can be used in order to construct other modified antibody molecules.

Suitable vectors for the cloning and subcloning steps used in the methods and construction of the compositions provided herein can be selected by a person skilled in the art. For example, the conventional pUC series of cloning vectors can be used. One vector, pUC19, is commercially available from suppliers such as Amersham (Buckinghamshire, United Kingdom) or Pharmacia (Uppsala, Sweden). In addition, any vector which can be easily multiplied, has a multiplicity of cloning sites and selectable genes (e.g., antibiotic resistance) and is easily manipulated can be used for cloning.

Similarly, the vectors used for the expression of the antibodies can be selected from any conventional vector by a person skilled in the art. The vectors also contain selected regulatory sequences (such as CMV promoters) which achieve the multiplication and expression of heterologous DNA sequences in selected host cells. Said vectors contain the above-described DNA sequences which encode the antibody or the modified immunoglobulin-coding region. In addition, the vectors can accept the selected immunoglobulin sequences which have been modified for easy manipulation by the insertion of desired restriction sites.

The expression vectors can also be characterized by genes which are suitable for amplifying the expression of the heterologous DNA sequences, for example the mammalian dihydrofolate reductase gene (DHFR). Other preferred vector sequences include a poly A signal sequence, such as, for example, from bovine growth hormone (BGH); and the beta-globin promoter sequence (Betaglopro). The expression vectors useful here can be synthesized by techniques commonly known to those skilled in the art.

The components of such vectors, for example replicons, selection genes, enhancers, promoters, signal sequences and the like, can be obtained from commercial or natural sources or be obtained by known methods for use in achieving the expression and/or secretion of the product of the recombinant DNA in a selected host. Other suitable expression vectors, numerous types of which are known in this field for mammalian, bacterial, insect, yeast and fungal expression, can likewise be selected for this purpose.

The vectors can be used for generation of a cell line which has been transfected with a recombinant plasmid which contains the coding sequences of the antibodies or modified immunoglobulin molecules thereof. Host cells useful for cloning and other manipulations of these cloning vectors are likewise conventional. For example, bacterial cells, such as cells from various strains of E. coli, yeast cells, insect cells or mammalian cells can be used for multiplication of the cloning vectors and for other steps in the construction of modified antibodies of this invention.

Suitable host cells or cell lines for expression of the antibody of the invention are preferably mammalian cells such as NS0, Sp2/0, CHO, COS, a fibroblast cell (e.g., 3T3) and myeloid cells and particularly preferably a CHO or myeloid cell. Human cells can be used, thereby making it possible to modify the molecule with human glycosylation patterns. Alternatively, other eukaryotic cell lines can be used. The selection of suitable mammalian host cells and methods for transformation, culturing, amplification, screening and product production and purification are known in the art; see, for example, Sambrook et al., cited above.

Bacterial cells may prove useful as host cells suitable for the expression of the recombinant Fabs of the present invention (see, for example, A. Plückthun, Immunol. Rev., 130: 151-188 (1992)). However, because of the tendency of proteins expressed in bacterial cells to be in an unfolded or inappropriately folded form or in a nonglycosylated form, any recombinant Fab generated in a bacterial cell would have to be screened for retention of antigen-binding ability. If the molecule expressed by the bacterial cell were to be generated in an appropriately folded form, the bacterial cell would be a desirable host. For example, various strains of E. coli used for expression are commonly known as host cells in the field of biotechnology.

Various strains of B. subtilis, Streptomyces, other Bacilli and the like can also be used in this method.

As required, strains of yeast cells known to those skilled in the art are also available as host cells, as are insect cells, for example Drosophila and Lepidoptera, and viral expression systems. See, for example, Miller et al., Genetic Engineering, 8: 277-298, Plenum Press (1986) and references cited therein.

The general methods by which the vectors can be constructed, the transfection methods which are necessary for generation of the host cells, and culturing methods which are necessary for generation of the modified antibody of the invention from such host cells are all conventional techniques. Similarly, once the antibodies of the invention have been generated, they can be purified from the cell culture contents according to standard methods in this field, which include ammonium sulfate precipitation, affinity columns, column chromatography, gel electrophoresis and the like. Such techniques are within the ability of a person skilled in the art.

Once expressed by the desired method, the antibody is then tested for in vitro activity by use of an appropriate test (assay). Conventional ELISA test formats are currently used for assessing the qualitative and quantitative binding of the antibody to MAG. In addition, it is possible to use other in vitro assays for verification of specific binding to the antigen and neutralizing efficacy as described herein prior to subsequent human clinical studies, which are conducted to evaluate the persistence of the antibody in the body despite the usual clearance mechanisms.

In a preferred embodiment, the antibody or the antigen-binding fragment is chosen from the group consisting of: immunoglobulin molecule, polyclonal antibody, monoclonal antibody, chimeric antibody, antibody generated by CDR-grafting, humanized antibody, Fab, Fab′, F(ab′)2, Fv, disulfide-linked Fv, scFv, single-domain antibody, diabody, multispecific antibody, dual-specific antibody and bispecific antibody.

Preferably, the antibody is a chicken immunoglobulin molecule or antibody. The antibody is preferably generated against an antigen or suitable peptide of the bacterium Candidatus savagella and is preferably a polyclonal or monoclonal chicken antibody. At the same time, the chicken antibody can be humanized or modified, as defined elsewhere.

Particularly preferably, the chicken immunoglobulin molecule is an IgY immunoglobulin molecule or polyclonal IgY antibody.

Immunoglobulin Y, abbreviated as IgY, is a class of immunoglobulin molecules which are present in the serum of chickens and, in high concentrations, especially in the yolks of chicken eggs. As with the other immunoglobulins, IgYs are also proteins which are formed by the immune system in response to certain foreign structures and specifically recognize them.

Immunoglobulin Y is the functional equivalent of IgG in chickens and, like IgG, is made up of two light and two heavy chains. Structurally, the two immunoglobulin classes differ especially in the heavy chains, which have a molecular mass of about 65.1 kilodaltons in the case of IgY and are hence larger than in the case of IgG. With a molar mass of about 18.7 kilodaltons, the light chains of IgY are slightly smaller compared to IgG. The molar mass of IgY is hence about 167 kilodaltons. The steric flexibility of the IgY molecule is less than that of IgG.

Functionally, IgY is comparable in part to both IgE and IgG. However, in contrast to IgG, IgY binds neither to protein A or protein G nor to cellular Fc receptors. Furthermore, IgY does not activate the complement system. The name immunoglobulin Y was proposed in 1969 by G. A. Leslie and L. W. Clem after they were able to show differences between the immunoglobulins found in chicken eggs and immunoglobulin G.

For the specific recovery of antibodies and the use thereof in bioanalytics, IgY offers various advantages over the use of mammalian antibodies. Since the antibodies are recovered from the yolk of laid eggs, a noninvasive method of antibody production is concerned. Thus, no blood has to be withdrawn from the chickens in order to obtain blood serum. The repeated laying of eggs by the same chicken considerably increases the available amount of a particular antibody. The cross-reactivity with mammalian proteins is, too, distinctly lower than that of IgG. Furthermore, the immune response to particular antigens is more pronounced in chickens than in rabbits or other mammals. Since, of the immunoglobulins formed during the immune response, only IgY can be found in chicken eggs, corresponding preparations do not contain IgA or IgM contamination. The yield of IgY from one chicken egg is high and comparable to that of IgG from rabbit serum. Since IgY does not bind to protein A/G, does not activate complement factors and does not bind to the Fc part of antibodies, the result is a reduced background in many applications.

Furthermore, IgY is used as a food constituent, especially in Asian countries such as Japan. For example, yoghurt products containing specific IgY are sold there. It prevents the adhesion of bacteria of the species Helicobacter pylori in the stomach. The IgY used for this purpose is recovered from the eggs of immunized chickens. Antibodies are thus also produced against Salmonellae and other bacteria, but also against viruses, and used as a food constituent for protection against these pathogens. Thus, the IgY according to the invention can, for example, be used in the form of novel food or nutraceuticals.

There are now a number of commercial suppliers for the production of polyclonal antibodies from chicken egg yolk or the production of monoclonal antibodies from chickens (e.g., Genosphere Biotechnologies, David's Biotechnology).

In a further preferred embodiment, the antibody or the antigen-binding fragment (indirectly) leads to a reduction in Th17 cell proliferation, Th17 cell differentiation or Th17 cell activity. Alternatively or additionally, the antibody or the antigen-binding fragment inhibits the formation of antibodies against endogenous antigens by B cells.

Th17 cells develop from so-called naive T helper cells, i.e., T helper cells which have not yet had any contact with their specific antigen, only after activation thereof by antigen contact. The differentiation of Th17 cells requires IL-6 and TGF-β. Th17 cells are named after the interleukin IL-17 they produce, and play an important role in the activation of neutrophils, but are also associated with the development of chronic inflammations and autoimmune diseases. Further secretion products from Th17 cells are TNF-α and IL-6. Receptors for IL-17 are located on various cell types of the immune system, for example on myeloid cells, which include neutrophils, and on lymphocytes. The messenger substances released by the Th17 cells cause inflammatory processes, in which neutrophils play a dominant role. In the target cells mentioned, IL-17 causes a release of, inter alia, G-CSF, IL-6 and IL-8, which cause migration of neutrophils and activation thereof. In addition, proinflammatory proteins such as IL-1, IL-6, PGE-2, cyclooxygenase 2 and matrix metalloproteases are expressed to an increased extent.

As a result of the above-mentioned mechanisms, i.e., reduction of Th17 cell proliferation, Th17 cell differentiation or Th17 cell activity and/or inhibition of the formation of antibodies against endogenous antigens by B cells, the antibody of the invention contributes to an increased immunotolerance. Immune diseases such as multiple sclerosis, rheumatoid arthritis, type 1 diabetes and allergic asthma are substantially determined by the activity of Th17 immune cells. The intestinal bacterium Candidatus savagella (segmented filamentous bacteria) induces precisely these Th17 immune cells (see, for example, US 2012/276149; WO 2011/047153). The present invention encompasses the neutralization of said bacterium with orally administered chicken protein antibodies in order to therapy Th17 immune cell-mediated diseases. Tests for reduction of Th17 cell proliferation, reduction of Th17 cell differentiation, reduction of Th17 cell activity or inhibition of the formation of antibodies against endogenous antigens by B cells are known to a person skilled in the art and are described in the prior art; see, for instance, US 2012/276149 or WO 2011/047153.

In a preferred embodiment, the antigen of the bacterium Candidatus savagella is a bacterial wall protein, particularly preferably the myosin-cross-reactive antigen (SEQ ID No. 1).

An example of an antigen in the context of the invention is a cell wall protein of the bacterium Candidatus savagella, such as, for instance, the myosin-cross-reactive antigen, the amino acid sequence of which is shown in SEQ ID NO. 1.

In a preferred embodiment, the antibody or the antigen-binding fragment binds to an epitope from the myosin-cross-reactive antigen, comprising or consisting of the amino acid sequence SVLDEFYWLDKKDPYSL (SEQ ID No. 2), PDFKAVRFTRRNQYESMI (SEQ ID No. 3) or QATSIKILRDGKEEEIKL (SEQ ID No. 4).

The invention also relates to a method for producing an antibody according to the invention, comprising:

a) immunizing chickens with an immunogenic peptide from an antigen of the bacterium Candidatus savagella; and
b) recovering and purifying the antibodies formed in the chickens or in an egg laid by said chickens.

A neutralizing antibody of the invention is generated by selecting, from the group of function-determining and segmented filamentous bacteria (SFB)-specific proteins, a suitable protein which is characterized in that it is a bacterial wall protein and plays a nonredundant role in adherence to the intestinal epithelium and/or the survival of Candidatus savagella or segmented filamentous bacteria (SFB), such as, for example, the myosin-cross-reactive antigen (MCRA) protein. Suitable epitopes are then determined within the protein sequence of the selected target antigen, for example by means of epitope prediction programs and/or with the aid of databases. The target sequence having the mathematically best antigenicity/immunogenicity is then selected for antibody production. Using the amino acid sequence, the corresponding peptide is synthesized in a sufficient amount in the next step. In order to be able to act as an antigen, low-molecular-weight molecules such as peptides must be coupled to carrier proteins (carriers). In addition to ovalbumin (chicken egg albumin) and bovine or human serum albumins, the snail protein KLH is a carrier protein widespread in biotechnology for the immunization of animals. Thus, the peptide can then, for example, be coupled to the protein keyhole limpet hemocyanin (KLH). The chickens are immunized four times with the peptide-KLH complex within 60 days, and during the first immunization the animal receives twice the amount. After this immunization phase, one chicken will produce up to 3 grams of IgY per month. The antibodies are then isolated from the egg yolk and tested for their neutralizing effect, i.e., whether the antibody is capable of inhibiting the adhesion or binding of the bacterium Candidatus savagella to intestinal epithelial cells, of inhibiting the proliferation of said bacterium and/or of depleting or killing the bacterium.

What is preferred for producing the antibody according to the invention by the above-mentioned method is the myosin-cross-reactive antigen as antigen of the bacterium Candidatus savagella. Particular preference is given to the epitope sequences from the myosin-cross-reactive antigen having the amino acid sequence SEQ ID No. 1, i.e., peptides having the amino acid sequence SVLDEFYWLDKKDPYSL (SEQ ID No. 2), PDFKAVRFTRRNQYESMI (SEQ ID No. 3) or QATSIKILRDGKEEEIKL (SEQ ID No. 4).

The definitions and explanations of the terms given above apply mutatis mutandis to the embodiments described below.

The present invention also relates to a drug comprising the antibody or an antigen-binding fragment of the invention or the antibody produced by the method according to the invention. Preferably, the antibody or an antigen-binding fragment of the invention is suspended in a pharmaceutically acceptable carrier. Preferably, the patient to be treated is a human.

A further subject of the present invention is a drug comprising the antibody or an antigen-binding fragment of the invention or the antibody produced by the method according to the invention and also preferably suitable additives and/or excipients. Suitable additives and/or excipients are, for example, a physiological saline solution, suitable stabilizers, proteinase inhibitors, etc. Suitable stabilizers are, for example, Tween 80 (0.02%), sugar solutions, such as, for example, sucrose solution (20-30%), or amino acid solutions, such as, for example, glycine or cysteine solutions. The drug according to the invention is usually produced by admixing of the antibody or an antigen-binding fragment of the invention with suitable additives and/or excipients.

The therapeutic agents of this invention can be administered as a prophylactic or otherwise as needed. The dose and duration of treatment is related to the relative duration of the molecules of the present invention in the human circulation and can be adjusted by a person skilled in the art depending on the treated condition and the general health of the patient.

The mode of administration of the therapeutic agent of the invention can be any suitable route that will deliver the agent to the host. The antibodies and pharmaceutical compositions/drugs of the invention are particularly useful for oral administration.

In a preferred embodiment, the drug according to the invention is for use for prevention or treatment of an oncological disease, an allergic disease or an immune disease, preferably an autoimmune disease, said diseases being mediated by the activity of Th17 cells.

For example, an allergy sufferer or patient suffering from an autoimmune disease takes the specific SFB antibodies of the invention orally over a defined period of time in a defined treatment regimen. Then, the reduction in the allergic reaction is, for example, determined via a prick test. The effect of the specific SFB antibodies on a patient suffering from an autoimmune disease can, for example, be detected via a reduction in the formation of autoantibodies. The reference corresponds to the intensity of the reaction in the absence of SFB antibody treatment.

Preferably, the allergic disease, immune disease or autoimmune disease is selected from the group consisting of: multiple sclerosis, type 1 diabetes, rheumatoid arthritis, allergic respiratory disease and allergic asthma.

In a further preferred embodiment, the antibody or an antigen-binding fragment thereof is administered orally. Chicken antibodies (IgY) are particularly acid-stable and are therefore particularly suitable for an oral use in patients. However, all previous routes used for the transfer of immunoglobulins are also encompassed, for example intramuscular, intravenous, intraperitoneal or intrathecal administration.

In a further preferred embodiment, the antibody or an antigen-binding fragment thereof is used in combination with an antibiotic, preferably a beta-lactam and/or glycopeptide antibiotic.

The present invention moreover relates to a method for producing a drug according to the invention, comprising:

a) producing an antibody according to the invention or an antigen-binding fragment thereof; and
b) formulating the antibody or an antigen-binding fragment thereof as a drug.

The present invention lastly relates to a kit comprising an antibody according to the invention or an antigen-binding fragment thereof for reduction of Th17 cell proliferation, Th17 cell differentiation or Th17 cell activity and/or inhibition of the formation of antibodies against endogenous antigens by B cells.

In addition to the antibody according to the invention or an antigen-binding fragment thereof, the kit can also comprise an antibiotic such as beta-lactam and/or a glycopeptide antibiotic and also instructions for using the components of the kit.

The term “about” as used herein refers to a specific value referred to in this description, including a variant in the range of +/−20%, +/−10%, +/−5%, +/−4%, +/−3%, +/−2% or +/−1%.

The terms “determine”, “determination” or “detection” as used here encompass qualitative, semiquantitative and/or quantitative determination, for example the quantitative determination of the antibody of the invention in the serum or plasma of a patient to whom said antibody was administered for therapeutic purposes.

The content of all literature references cited herein is hereby incorporated by reference to the respective specific disclosure content and in the entirety thereof.

Further particularly preferred embodiments of the invention are shown in the following examples.

### FIGURES

FIG. 1: Graphic summary of the concept forming the basis of this invention: Specific filamentous Candidatus savagella bacteria (segmented filamentous bacteria, SFB) colonize the intestinal wall and, via dendritic cells (DC), activate Th17 cells, which contribute to autoimmunity and atopy. First, suitable bacterial wall proteins of said bacterium are identified (1), synthesized and injected into chickens (2). The chickens form highly specific anti-SFB antibodies, which can be isolated from the eggs (3) and are available for oral antibody therapy in humans (4). A reduction in the SFB microbial count in the intestines can lead to a reduction in Th17 effector cell activity and thus to immunotolerance.

### EXAMPLES

The following examples serve to illustrate the invention. With regard to the scope of protection, they must not be interpreted in a restrictive manner.

### Example 1: Production and Recovery of Neutralizing Antibodies Against the Myosin-Cross-Reactive Antigen (MCRA) Protein

From the group of function-determining and segmented filamentous bacteria (SFB)-specific proteins, what was selected was the myosin-cross-reactive antigen (MCRA) protein, which represents a suitable target for a neutralizing antibody owing to the bacterial-wall location and the nonredundant role in adherence to the intestinal epithelium in humans and the survival of Candidatus savagella or segmented filamentous bacteria (SFB). Suitable epitopes were then determined within the known protein sequence of the MCRA protein (SEQ ID No. 1). The target sequence having the arithmetically best antigenicity was selected for antibody production. In the next step, using the known amino acid sequence, the corresponding peptide having SEQ ID Nos. 2, 3 and 4 was synthesized in a sufficient amount and then coupled to the protein keyhole limpet hemocyanin (KLH). The chickens were immunized four times with the peptide-KLH complex within 60 days, and during the first immunization the animal received twice the amount. After this immunization phase, one chicken produced up to 3 grams of IgY per month. The antibodies were then isolated from the egg yolk (Hass) et al., 1987 http://www.unet.univie.ac.at/^{˜}a7505973/texte/JiM1.pdf).

### Example 2: Therapeutic Use of Neutralizing Antibodies Against the Myosin-Cross-Reactive Antigen (MCRA) Protein

According to the underlying working hypothesis of the inventors, a reduced SFB colonization density in the intestines of patients leads to a reduced TH17 response. This would generally be of huge therapeutic relevance in TH17-mediated inflammatory diseases.

It is assumed by the inventors that SFB also exists in the intestines of human patients and that oral therapy with a neutralizing antibody might be suitable for the treatment of a whole range of allergic diseases, immune diseases, autoimmune diseases and oncological diseases.

The therapeutic approach chosen is particularly attractive because hardly any adverse effects are to be expected—in contrast to most established therapies, such as, for example, dexamethasone for allergic respiratory disease—and there may be possibilities for a greatly simplified authorization procedure, for example as novel food.

In this regard, the use of chicken antibodies (IgY) is a basic concept of the present invention. The use of chicken antibodies (IgY) has numerous advantages over classic approaches: for example, IgYs do not react with the human complement system and have a particularly high acid stability, which makes them attractive for the development of an orally administered therapeutic.

The oral administration can, in this case, look as follows:

An allergy sufferer takes the specific SFB antibodies orally over a defined period of time in a defined treatment regimen. Then, the reduction in the allergic reaction is, for example, determined via a prick test. The reference corresponds to the intensity of the reaction in the absence of SFB antibody treatment.

In summary, the suitable SFB bacterial wall proteins were identified, synthesized and injected into chickens in this project. The chickens produced highly specific, neutralizing anti-SFB antibodies that were isolated from the eggs. This product isolated from the eggs and having pharmaceutical potential can then be used for oral antibody therapy in patients for treatment of a whole range of allergic diseases, immune diseases, autoimmune diseases and oncological diseases.

### LITERATURE CITED

[1] Cerf-Bensussan N, Gaboriau-Routhiau V: The immune system and the gut microbiota: friends or foes? Nat Rev Immunol 2010; 10: 735-744.
[2] Lee Y K, Menezes J S, Umesaki Y, Mazmanian S K: Proinflammatory T-cell responses to gut microbiota promote experimental autoimmune encephalomyelitis. Proc. Natl Acad Sci-U-S-A 15 Mar. 2011; 108 Suppl 1: 4615-4622.
[3] Wu H J, Ivanov I I, Darce J, Hattori K, Shima T, Umesaki Y, Littman D R, Benoist C, Mathis D: Gut-residing segmented filamentous bacteria drive autoimmune arthritis via T helper 17 cells. Immunity 25 Jun. 2010; 32: 815-827.
[4] http://www.atsjournals.org/doi/abs/10.1164/ajrccm-conference. 2012.185.1_MeetingAbstracts.A 1444
[5] Spillner E, Braren 1, Greunke K, Seismann H, Blank S, du P D: Avian lgY antibodies and their recombinant equivalents in research, diagnostics and therapy. Biologicals 2012; 40: 313-322.
[6] Wills-Karp M, Santeliz J, Karp C L: The germless theory of allergic disease: revisiting the hygiene hypothesis. Nat Rev Immunol 2001; 1: 69-75.
[7] Bach J F: The effect of infections on susceptibility to autoimmune and allergic diseases. N Engl J Med 19 Sep. 2002; 347: 911-920.
[8] Atarashi K, Tanoue T, Oshima K, Suda W, Nagano Y, Nishikawa H, Fukuda S, Saito T, Narushima S, Hase K, Kim S, Fritz J V, Wilmes P, Ueha S, Matsushima K, Ohno H, Olle B, Sakaguchi S, Taniguchi T, Morita H, Hattori M, Honda K: Treg induction by a rationally selected mixture of Clostridia strains from the human microbiota. Nature 8 Aug. 2013; 500: 232-236.
[9] Wang H, Geier M S, Howarth G S: Prebiotics: a potential treatment strategy for the chemotherapy-damaged gut? Crit Rev Food Sei Nutr 27 Aug. 2014.
[10] Zeissig S, Blumberg R S: Life at the beginning: perturbation of the microbiota by antibiotics in early life and its role in health and disease. Nat Immunol 2014; 15: 307-310.
[11] Lecuyer E, Rakotobe S, Lengline-Garnier H, Lebreton C, Picard M, Juste C, Fritzen R, Eberl G, McCoy K D, Macpherson A J, Reynaud C A, Cerf-Bensussan N, Gaboriau-Routhiau V: Segmented filamentous bacterium uses secondary and tertiary lymphoid tissues to induce gut lgA and specific T helper 17 cellresponses. lmmunity 17 Apr. 2014; 40: 608-620.
[12] Goto Y, Panea C, Nakato G, Cebula A, Lee C, Diez M G, Laufer T M, lgnatowicz L, lvanov II: Segmented filamentous bacteria antigens presented by intestinal dendritic cells drive mucosal Th17 cell differentiation. lmmunity 17 Apr. 2014; 40: 594-607.
[13] lvanov II, Atarashi K, Manel N, Brodie E L, Shima T, Karaoz U, Wei D, Goldfarb K C, Santee C A, Lynch S V, Tanoue T, lmaoka A, ltoh K, Takeda K, Umesaki Y, Honda K, Littman D R: Induction of intestinal Th17 cells by segmented filamentous bacteria. Cell 30 Oct. 2009; 139: 485-498.
[14] Davidson G P, Whyte P B, Daniels E, Franklin K, Nunan H, McCloud P I, Moore A G, Moore D J: Passive immunisation of children with bovine colostrum containing antibodies to human rotavirus. Lancet 23 Sep. 1989; 2: 709-712.
[15] Tollemar J, Gross N, Dolgiras N, Jarstrand C, Ringden 0, Hammarstrom L: Fungal prophylaxis by reduction of fungal colonization by oral administration of bovine anti-Candida antibodies in bone marrow transplant recipients. Bone Marrow Transplant 1999; 23: 283-290.
[16] Shimamoto C, Tokioka S, Hirata 1, Tani H, Ohishi H, Katsu K: Inhibition of Helicobacter pylori infection by orally administered yolk-derived anti-Helicobacter pylori antibody. Hepatogastroenterology 2002; 49: 709-714.
[17] Torres 0, Cruz J R: Protection against Campylobacter diarrhea: role of milk lgA antibodies against bacterial surface antigens. Acta Paediatr 1993; 82: 835-838.
[18] Ochi H, Abraham M, lshikawa H, Frenkel D, Yang K, Basso A S, Wu H, Chen M L, Gandhi R, Miller A, Maron R, Weiner H L: Oral CD3-specific antibody suppresses autoimmune encephalomyelitis by inducing CD4+. Nat Med 2006; 12: 627-635.

1. An antibody or an antigen-binding fragment thereof, wherein the antibody or the antigen-binding fragment is configured to bind to an antigen of the bacterium Candidatus savagella and (i) inhibits the adhesion of the bacterium to intestinal epithelial cells, preferably human intestinal epithelial cells, and/or (ii) depletes the bacterium.

2. The antibody or an antigen-binding fragment thereof as claimed in claim 1, wherein the antibody or the antigen-binding fragment is selected from the group consisting of: immunoglobulin molecule, polyclonal antibody, monoclonal antibody, chimeric antibody, antibody generated by CDR-grafting, humanized antibody, Fab, Fab′, F(ab′)₂, Fv, disulfide-linked Fv, scFv, single-domain antibody, diabody, multispecific antibody, dual-specific antibody and bispecific antibody.

3. The antibody or an antigen-binding fragment thereof as claimed in claim 1, wherein the antibody is a chicken immunoglobulin molecule or antibody.

4. The antibody or an antigen-binding fragment thereof as claimed in claim 3, wherein the chicken immunoglobulin molecule or antibody is IgY.

5. The antibody or an antigen-binding fragment thereof as claimed in claim 1, wherein the antibody or the antigen-binding fragment leads to a reduction in Th17 cell proliferation, Th17 cell differentiation or Th17 cell activity and/or inhibits the formation of antibodies against endogenous antigens by B cells.

6. The antibody or an antigen-binding fragment thereof as claimed in claim 1, wherein the antigen of the bacterium Candidatus savagella is a bacterial wall protein.

7. The antibody or an antigen-binding fragment thereof as claimed in claim 1, wherein the antibody or the antigen-binding fragment binds to an epitope from the myosin-cross-reactive antigen.

8. A method for producing an antibody as claimed in claim 1, the method comprising:
a) immunizing chickens with an immunogenic peptide from an antigen of the bacterium Candidatus savagella; and
b) recovering and purifying the antibody formed in the chickens or in an egg laid by said chickens.

9. A drug comprising the antibody or an antigen-binding fragment thereof as claimed in claim 1.

10. A method for prevention or treatment of an oncological disease, an allergic disease, an immune disease or an autoimmune disease, said diseases being mediated by the activity of Th17 cells, the method comprising administering the as claimed in claim 9.

11. The drug as claimed in claim 10, wherein the allergic disease, immune disease, or autoimmune disease is selected from the group consisting of multiple sclerosis, type 1 diabetes, rheumatoid arthritis and allergic asthma.

12. The drug as claimed in claim 9, wherein the antibody or an antigen-binding fragment thereof is configured to be administered orally.

13. The drug as claimed in claim 9, wherein the antibody or an antigen-binding fragment thereof is configured to be used in combination with an antibiotic.

14. A method for producing a drug as claimed in claim 9, comprising:
a) producing an antibody or an antigen-binding fragment thereof configured to bind to an antigen of the bacterium Candidatus savagella and (i) inhibits the adhesion of the bacterium to intestinal epithelial cells, preferably human intestinal epithelial cells, and/or (ii) depletes the bacterium; and
b) formulating the antibody or an antigen-binding fragment thereof as a drug.

15. A kit comprising an antibody or an antigen-binding fragment thereof as claimed in claim 1, the kit being configured for reduction of Th17 cell proliferation, Th17 cell differentiation or Th17 cell activity and/or inhibition of the formation of antibodies against endogenous antigens by B cells.

16. The antibody or an antigen-binding fragment thereof as claimed in any of claim 6, wherein the myosin-cross-reactive antigen comprising the amino acid sequence shown in SEQ ID No. 1.

17. The antibody or an antigen-binding fragment thereof as claimed in claim 7, wherein the epitope comprises the amino acid sequence SVLDEFYWLDKKDPYSL (SEQ ID No. 2), PDFKAVRFTRRNQYESMI (SEQ ID No. 3), and/or QATSIKILRDGKEEEIKL (SEQ ID No. 4).

18. The method as claimed in claim 8, wherein the antigen is a bacterial wall protein of the bacterium Candidatus savagella.

19. The drug as claimed in claim 13, wherein the antibiotic is a beta-lactam and/or a glycopeptide antibiotic.

20. A kit as claimed in claim 15, wherein the kit further comprises an antibiotic.


SEQUENCE LISTING (4 sequences)

SEQ ID NO: 1 - protein, 591 aa, Candidatus savagella
  Myosin-cross-reactive antigen
MYYSSGNYEAFARPIKPEGVDNKSAYLIGSGLASLVAAFYLVRDAQMKGENIHILEKDSIPGGACDGFDYKGIGYVMRGGREIDNHYEILWDVFRSVPSIEIEGASVLDEFYWLDKKDPNYSLRRATVNRGEDAKMENKFGLSDKASLQIMRLFFTPDEDLYDKPITDYFDDEVLKSNFWLYWRTMFAFQNWHSALEMKLYIKRFIHHIGGMPDFKAVRFTRRNQYESMILPLIKYLESHNVDFQYNTKVVDVEFDIQPHKKQATSIKILRDGKEEEIKLTENDLLFITNGGCVENSSIGSQNTPAVLNSEIKEGGGWDMWRRISVKDPSFGNPDKFCYDAEKSNWMSATVTVLDDKIVPYIKNICQRDPFSGKVVTGGIVTVKDSKWLLSWTFNRQPQFREQPKGQLVGWIYGLFSDVAGDYIKKPMRDCTGKEICMEWLYHLGVPEDQIVDLAENHANTVPCMMPYITAFFMPRSKGDRPNVVPTGSVNFAFIGQFAETERDTIFTIEYSARTGIESVYTLLNVDRGVPEVWGSTFDIRDLVNATYCMRDGKKITDMKLGFKENIALKELLKKISGTDVEKLLKEYKLI

SEQ ID NO: 2 - protein, 17 aa, Artificial Sequence
  Target 1
SVLDEFYWLDKKDPYSL

SEQ ID NO: 3 - protein, 18 aa, Artificial Sequence
  Target 2
PDFKAVRFTRRNQYESMI

SEQ ID NO: 4 - protein, 18 aa, Artificial Sequence
  Target 3
QATSIKILRDGKEEEIKL